(19) United States
(12) Patent Application Publication
(10) Pub. No.: US 2025/0312513 A1
(43) Pub. Date: Oct. 9, 2025

(54) MOLDABLE, STRETCHABLE, AND SELF-HEALING HYDROGEL ADHESIVES

(71) Applicant: THE TRUSTEES OF THE UNIVERSITY OF PENNSYLVANIA, Philadelphia, PA (US)

(72) Inventors: Shu YANG, Blue Bell, PA (US); Mingtao CHEN, Sichuan (CN); Baohong CHEN, Philadelphia, PA (US)

(21) Appl. No.: 18/867,473

(22) PCT Filed: May 25, 2023
(86) PCT No.: PCT/US2023/067474

§ 371 Date: Nov. 20, 2024

Related U.S. Application Data
(60) Provisional application No. 63/365,286, filed on May 25, 2022.

(51) Int. Cl. A61L 24/04; A61L 24/02

(52) U.S. Cl. CPC A61L 24/043; A61L 24/02

(57) ABSTRACT

Cell engineering, soft robotics and wearable electronics often desire soft materials that are easy to deform, self-heal, and relax stress. Hydrogel, a type of hydrophilic networks, which can be made responsive to environmental stimuli, are often used in the aforementioned applications. However, conventional hydrogels often suffer from poor stretchability and repairability. Here, we report hydrogels consisting of boronic ester dynamic covalent bonds in a double network of poly(vinyl alcohol) together with chitosan, demonstrating extreme stretchability (up to 310 times of the original length), instant self-healing (within 5 sec), reusability as well as inherent adhesion. Their instant stress relaxation as a result of low activation energy of the boronic ester bond exchange (less than 20 KJ/mol) contributes to the extreme stretchability and self-healing behaviors. The hydrophilic environment readily incorporates various additives and provide opportunities in conductive soft materials, bio-signaling, and soft artificial joint.

### CROSS-REFERENCE TO RELATED APPLICATIONS

The present application claims priority to and the benefit of U.S. patent application No. 63/365,286, “Moldable, Stretchable, And Self-Healing Hydrogel Adhesives” (filed May 25, 2022). All foregoing applications are incorporated herein by reference in their entireties for any and all purposes.

### TECHNICAL FIELD

The present disclosure relates to the field of hydrogels and to the field of adhesive materials.

### BACKGROUND

Cell engineering, soft robotics and wearable electronics often desire soft materials that are easy to deform, self-heal, and relax stress. Hydrogel, a type of hydrophilic networks, which can be made responsive to environmental stimuli, are often used in the aforementioned applications. However, conventional hydrogels often suffer from poor stretchability and repairability. Accordingly, there is a long-felt need in the art for improved hydrogel compositions.

### SUMMARY

In meeting the described long-felt needs, the present disclosure provides a reversibly adhesive composition, comprising: a first network that comprises at least two hydroxyl-bearing chains of a first polymer, the at least two hydroxyl-bearing polymer chains being crosslinked by crosslinks that comprise one or more boronic ester bonds, a second network that comprises at least two hydroxyl-bearing chains of a second polymer, and the second polymer optionally hydrogen bonding to the first polymer.

Also provided is a method, comprising contacting a composition according to the present disclosure (e.g., according to any one of Aspects 1-27) to an adherend so as to adhere the composition to the adherend.

Further disclosed is a method, comprising hydrating a composition according to the present disclosure (e.g., according to any one of Aspects 1-27) that is adhered to an adherend so as to reduce adhesion between the composition and the adherend.

Also provided is a method, comprising contacting a first and second portion of a composition according to the present disclosure (e.g., according to any one of Aspects 1-27) so as to effect adhesion between the two portions and form a combined portion, optionally wherein the combined portion exhibits at least one of a modulus and a stress relaxation profile that is substantially identical to the at least one of a modulus and a stress relaxation profile of the first portion or the second portion.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1a-1b. Illustration of AB and ABC hydrogels.

FIGS. 2a-2g. Illustration of the extreme stretchability and instant self-healing of the disclosed compositions.

FIGS. 3a-3f. Illustration of the fast stress relaxation and low boronic ester bond exchange activation energy.

FIGS. 4a-4f. Illustration of conductive ABC hydrogels with SWCNTs.

FIGS. 5a-5b. Illustration of tensile tests of (a) AB₁₅C₂ and AB₅C₂ showed (b) no further enhancement from boric acid crosslinker concentration on hydrogel mechanical properties. Pulling rate: 40 mm/min.

FIGS. 6a-6b. Illustration of tensile tests of ABC hydrogels with various chitosan concentration at (a) 40 mm/min and (b) 300 mm/min pulling rate.

FIGS. 7a-7b. Comparison of the (a) tensile properties of AB₁₅C₂ (b) before and (c) after self-healing for 5 s.

FIG. 8. Dehydration tests of AB₁₅C₂ hydrogels in an open (blue circle) or closed (red square) environment. Open environment: ambient condition; closed environment: scintillation vial with closed cap. The solid lines are to guide the eyes.

FIGS. 9a-9b. Stress relaxation of AB₁₅ (a) under various initial strains and (b) consecutive relaxation cycles (1% initial strain).

FIG. 10. Eyring plot of AB₁₅C₂ and AB₁₅ generated from stress relaxation data to calculate dissociation enthalpy and entropy.

FIGS. 11a-11d. Stress relaxation of AB₁₅C₂ hydrogels with (a) 0.1 wt %, (b), 0.5 wt %, (c) 1 wt %, and (d) 2 wt % of SWCNTs at 303 K, 313 K, 323 K, and 333 K.

FIGS. 12a-12d. Arrhenius equation fitting of AB₁₅C₂ hydrogels with (a) 0.1 wt %, (b), 0.5 wt %, (c) 1 wt %, and (d) 2 wt % of SWCNTs to calculate boronic ester bond exchange activation energy. Dotted line: linear fitting.

FIG. 13. Shear storage modulus (G′) as a function of SWCNT loadings in AB₁₅C₂ hydrogels.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints.

For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Soft materials that are malleable, reusable, and self-healable while exhibiting excellent stretchability, good adhesion, and fast stress relaxation are highly desired for applications including tissue engineering, soft robotics, and wearable electronics. It remains an enormous challenge for any material to satisfy all the requirements. Elastomers, such as vulcanized rubbers, offer excellent stretchability but lacks re-processability due to the formation of a network of irreversible covalent bonds. On the contrary, thermoplastics can deform and flow when heated above the melting temperature but remains rigid at the room temperature. Thermoplastic elastomers (TPEs) that combine the processibility of thermoplastics and the elasticity of elastomers are attractive as tough yet stretchable and reprocessible materials.¹ Nevertheless, many are not repairable and often require synthesis of block copolymers.

Reversibility of the covalently crosslinked networks can potentially integrate stretchability, self-healing, and reprocessability through the introduction of non-covalent interactions or dynamic covalent bonds (DCBs). The former involves hydrogen bonding,² Coulombic interactions,³ chain entanglement,⁴ and supramolecular interactions,⁵ featuring weak bond energy (comparing to covalent bond), long healing time, slow stress relaxation, or dependence on external stimuli.⁶⁻⁸ DCBs, on the other hand, offer structural stability yet can undergo debonding and rebonding when activated. Therefore, they are attractive for self-healing and reusability.⁹ Commonly studied DCBs include ester,¹⁰ disulfide,¹¹ imine,¹² thiol-X,¹³ boronic ester,¹⁴ and silyl ether bond.¹⁵ Most DCBs, however, require high temperature, use of catalyst or external stimuli to achieve reversible bonding and debonding.⁹ Among them, boronic esters are readily reversible at the room temperature without use of any catalyst or external stimuli.⁹ Previous efforts primarily employed hydrophobic boronic acid (phenyl boronic acid, PBA) and catechol groups as the dynamic covalent crosslinker with elongation at break varying from 1.1 to 10 times of its original length, l_{o.}^{1, 16-21} Replacing PBA with boric acid (BA) allows hydrogels to achieve similar stretchability (λ=l/lₒ,˜7.5) and instant (within seconds) self-healing in the presence of alginates.²²

To address the brittleness of conventional hydrogels, double networks (DNs) of two physically entangled matrices have been synthesized. Network A is a flexible network with loosely crosslinked covalent or non-covalent interactions, and network B is a tough and durable network with permanently crosslinked covalent bonds. DNs exhibit high toughness (fracture energy up to 9000 J/m²) and good stretchability (10 to 20).^{3, 6, 23} However, DNs are typically not reprocessible.

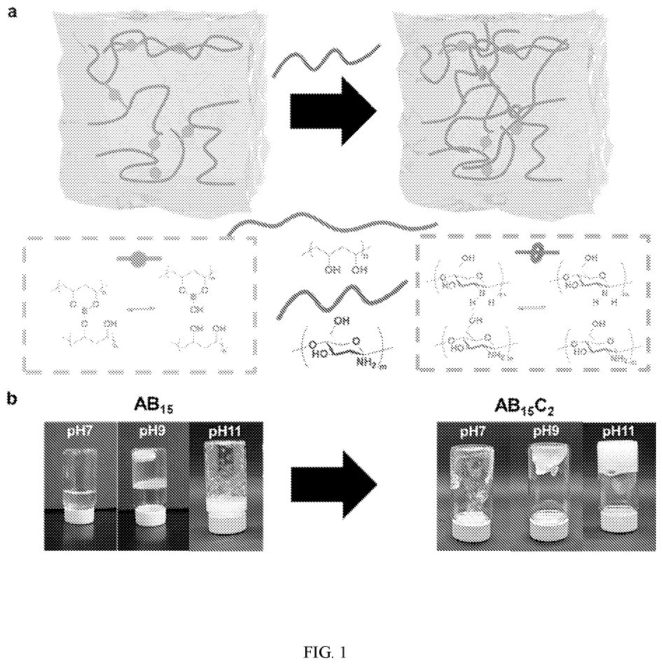

Here we integrated DCBs in DN hydrogel consisted of (A) dynamically crosslinked poly(vinyl alcohol) (PVA) and BA network and (B) chitosan, which is physically tethered with water via hydrogen bonding (FIG. 1a). Chitosan can also form hydrogen bonds with PVA to promote further entanglement between network A and B. The resulting PVA/BA/chitosan DN hydrogels, referred as ABC hydrogels, satisfied all the aforementioned criterions: extreme stretchability (>310), facile malleability (silly putty-like), good adhesion, reusability, spontaneous self-healing, and instant stress relaxation. Various hydrophilic additives (e.g., dyes, and carbon nanotubes) can be readily dissolved or dispersed in the ABC hydrogels to expand their applications in sensing and measurement.

The PVA/BA hydrogels (AB hydrogels) and the ABC hydrogels were prepared by mixing BA (and chitosan) solids with PVA solution (see Experimental Section). The nomenclature of AB₁₅ refers to a PVA/BA weight ratio of 15/1 while AB₁₅C₂ stands for a PVA/BA weight ratio of 15/1 and 2 wt % chitosan (relative to the water content). The total weight of PVA and BA was kept constant at 20 wt % in water. The physical appearance of AB and ABC hydrogels altered significantly at different pH values. As seen in FIG. 1b, at pH 7, both AB and ABC hydrogels were liquids as debonding of boronic esters was favored. At pH 9, also pKa of BA, debonding of boronic esters was severely limited, and both AB and ABC hydrogels became silly putty-like solids. Further increasing pH to 11 turned AB gels into viscous liquid while the chitosan in ABC gels provided additional structural stability from the hydrogen bonding to retain its solid-like appearance (although softer than the ABC gels at pH 9). This is because in an aqueous environment, BA exits in four forms: the neutral boric acid (H₃BO₃), the charged monoborate anion (B(OH)₄³¹ ), triborate anion (B₃O₄(OH)₃⁻), and tetraborate anion (B₄O₅(OH)₄⁻).²⁴ The neutral H₃BO₃ favors debonding while the anion forms prefer bonding. At pH 7, 60% of BA is in the neutral form, thus both AB and ABC are liquids. The rest is almost exclusively triborates. At pH 9, the composition of neutral boric acid decreases significantly to 10%, leaving the rest 90% BA in anion forms (30% for each) to promote bonding and gel formation. Hence, the coexistence of all four species is critical to the formation of the silly putty-like hydrogels. At pH 11, neutral form disappears completely, and the monoborate anions became dominant (>90%). AB hydrogel reverted to liquids again, while ABC hydrogel remained as a gel even at pH 11, supporting the important role of chitosan to improve structural stability via formation of hydrogen bonding. We note that in the pH range we studied (7 to 11), chitosan was not protonated (pKa˜6.5), hence contribution of ionic interactions is negligible.

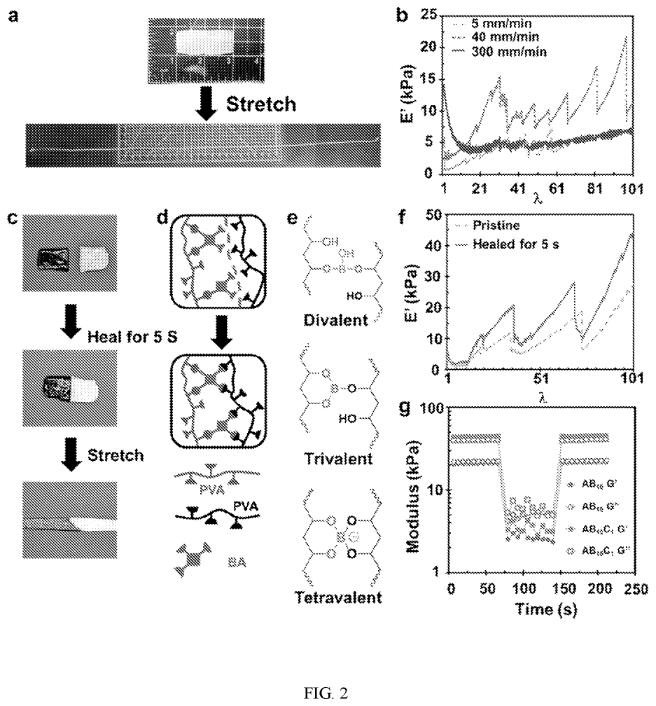

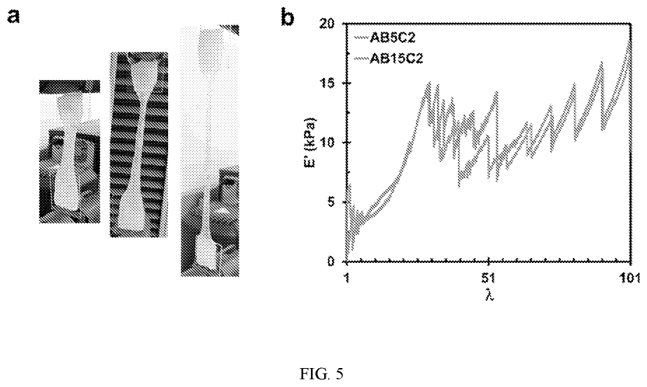

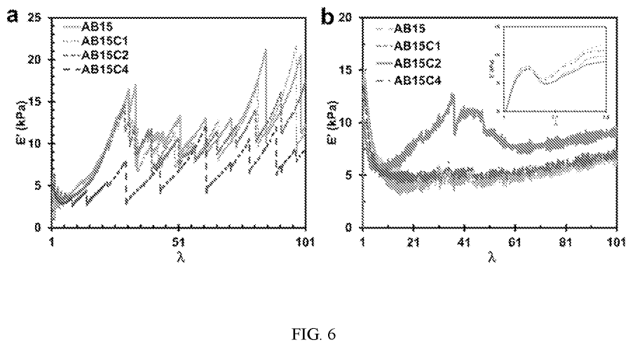

The ABC hydrogels demonstrated great malleability, good adhesion, and extreme stretchability. AB₁₅C₂ was easily deformed and conformed into a hexagonal mold. The hydrogel adhered to the mold and allowed effortless removal with no residual materials left on the mold. We also recorded an amazing elongation at break from 5.08 cm to 1575 cm, that is λ=310 (FIG. 2a), which to our best knowledge is the highest elongation value for hydrogels reported. To monitor the stress evolution during stretching, we applied uniaxial force to the ABC hydrogels (FIG. 2b). Various pulling rates were performed on AB₁₅C₂ (test sample preparation in Experimental Section, FIG. 5a). Altering the PVA/BA ratio, however, had negligible impact on the hydrogel mechanical properties (FIG. 5b), presumably due to the large amount of water (80%) in the network that mitigated the impact of the PVA/BA crosslinking density. At a pulling rate of 5 mm/min (I lₒ/min, l₀=5 mm), the stress-strain curves showed zig-zag profiles, implying a dynamic stress-relaxation during the tests, which will be discussed in detail later. The film broke at λ=70 due to the significant water evaporation and resulting hardening when the film was stretched very thin (FIG. 2b). When the pulling rate was increased to 40 mm/min, the hydrogel was stretched to the upper limit of the experiment (l=505 mm, λ=101) as there was less time for the thin film to dry and harden (FIG. 2b). To eliminate the influence of stress relaxation, an extremely fast pulling rate of 300 mm/min was employed, and the AB₁₅C₂ exhibited an early peak followed by a slow decay as seen in FIG. 2b. Under uniaxial force, AB₁₅C₂ proved to be the toughest materials compared with AB₁₅, AB₁₅C₁ and AB₁₅C₄ (FIG. 6) regardless of the pulling rate due to its highest activation energy for boronic ester bond exchange (see calculation later). Hence chitosan concentration dictated the mechanical properties of the ABC hydrogel. All AB and ABC hydrogels remained soft (<20 kPa) regardless of the pulling rates-an attribute that will be desired for wearable devices and tissue engineering.

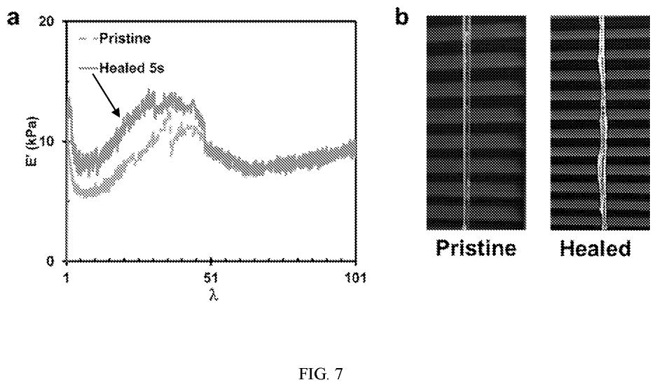

The hydrogels lost roughly 7% weight per day due to dehydration in an open environment (FIG. 7). However, when the hydrogels were stored in a closed scintillation vial, the water evaporation was negligible even after two weeks.

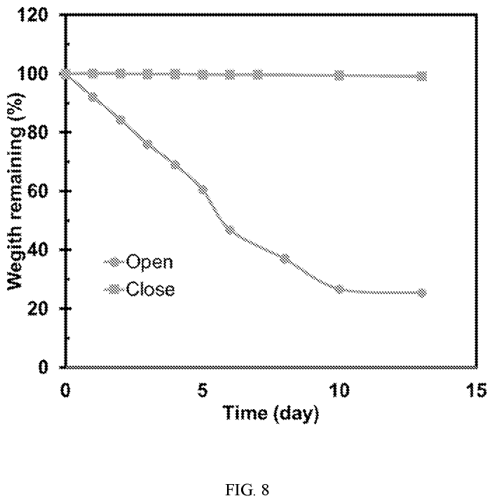

Instant self-healing occurred at the interfaces upon re-connecting two hydrogel pieces after cutting. Within seconds, the self-healing was completed, and no obvious cut boundary could be discerned. To better assist visualization of the self-healing process, two pieces of AB₁₅C₂ were prepared, and one of them was dyed with methylene blue (FIG. 2c). After making contacts for 5 s, the cut gels were spontaneously healed, leaving no trace of the boundary even under high strain (FIG. 2c, FIG. 2d). This is because at pH 9, 10% neutral BAs form divalent and trivalent boronic ester bonds (FIG. 2e) that are easy to break while 90% anionic BAs adopt tetravalent connection with low tendency towards debonding. AB₁₅C₂ showed compatible mechanical properties before and after healing under both slow (40 mm/min, FIG. 2f) and fast (300 mm/min, FIG. 8a) pulling rates. The slightly higher stress of the healed samples was ascribed to the heterogeneity during the stretch: the pristine samples exhibited uniform width at high strain, while the healed sample showed heterogeneous width (FIG. 8b). When oscillatory strain was applied, both AB and ABC hydrogels self-healed instantly as well. AB₁₅C₁ behaved like a solid (G′>G″) under 1% strain, and a sudden increase of strain (150%) ruptured the gel. Consequently, the gel liquified (G″<G′) (FIG. 2g). When the strain was returned to 1%, AB₁₅C₁ regained its solid-like behavior and recovered completely within 5 s. AB₁₅ showed a similar trend but started as a liquid (G′<G″) due to the absence of hydrogen bonding from chitosan (FIG. 2g).

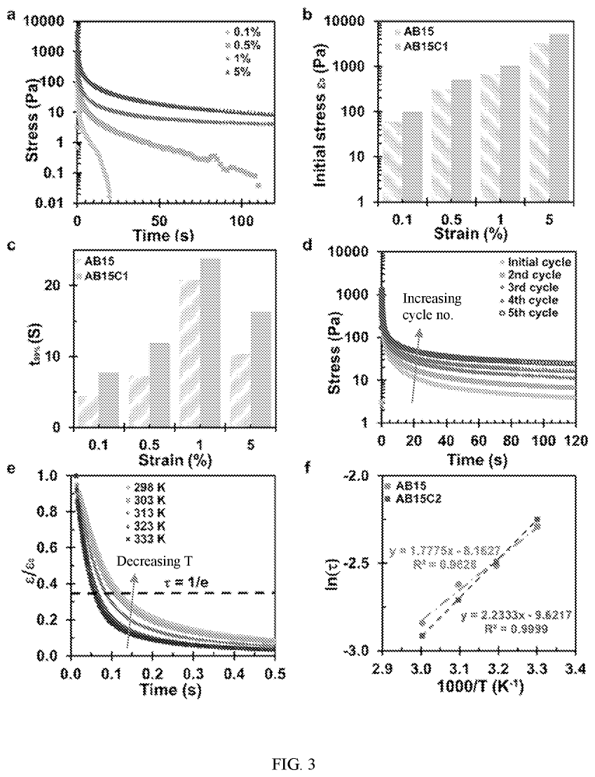

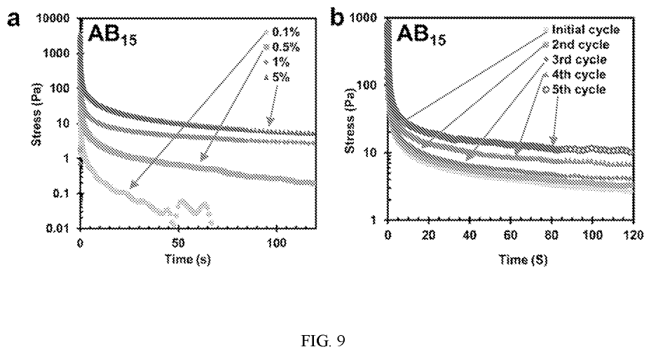

The extreme stretchability stemmed from the ultrafast stress relaxation of the boronic ester bonds. Both AB and ABC hydrogels exhibited instant stress relaxation under various initial strains (FIG. 3a and FIG. 9a).

TABLE 1
Summary of the calculated activation energy (Eₐ), dissociation enthalpy
$( Δ {H}_{diss}^{‡} ) , dissociation entropy ( Δ {S}_{diss}^{‡} ) , {τ}_{0} , and {t}_{99 \%} in AB and ABC hydrogels .$

| Hydrogel | Eₐ (KJ · mol⁻¹) | $Δ {H}_{diss}^{‡} ( kJ \cdot {mol}^{- 1} )$ | $Δ {S}_{diss}^{‡} ( \text{J/} {mol}^{- 1} \cdot {K}^{- 1} )$ | ε₀ (Pa) | t_{99%} (s) |
|---|---|---|---|---|---|
| AB₁₅ | 14.8 | 12.1 | −185.9 | 59 | 4.5 |
| AB₁₅C₁ | 16.7 | 14.1 | −179.5 | 102 | 7.8 |
| AB₁₅C₂ | 18.6 | 15.9 | −173.8 | 659 | 3.9 |
| AB₁₅C₄ | 16.4 | 13.8 | −178.4 | 580 | 2.8 |

The induced stress (ϵ₀) increased with increasing initial strain (FIG. 3b and Table 1). The time to complete 99% relaxation of the initial stress (t₉₉%) was under 25 s (FIG. 3c and Table 1). Consistently, AB₁₅C₁ responded to the applied strain with higher initial stress and relaxed slower than AB₁₅ since chitosan formed hydrogen bonds with water and PVA, providing extra stiffness (FIG. 3b and FIG. 3c). Consecutive stress relaxation induced by 1% initial strain showed a slight hysteresis for both AB₁₅ and AB₁₅C₁ (FIG. 3d and FIG. 9b). Nevertheless, more than 90% of the initial stress was dissipated within 20 s after 5 consecutive relaxation cycles. The instant stress relaxation allowed for efficient load dissipation within the hydrogels, leading to the extreme stretchability of ABC hydrogels shown in FIG. 2. When a defect was introduced to the AB₁₅C₂ gel, the extreme stretchability remained without any cut propagation owing to its ultrafast stress relaxation.

Activation energy (Eₐ) of the boronic ester bond exchange can be estimated:

$$τ ( T ) = {τ}_{0} exp ( \frac{{E}_{a}}{R T} ) \qquad ( 1 )$$

where τ(T) is the characteristic relaxation time at temperature T, R is the gas constant and Tis the temperature in K. τ(T) is defined as the characteristic relaxation time when 36.8% (1/e) of the initial stress is relaxed.²¹ A linear correlation between τ(T) and T was constructed through repeated stress relaxation process at different temperatures (FIG. 3e). Eₐ was then calculated from the slope of fitted linear correlation in FIG. 3f. As summarized in Table 1, Eₐ of AB and ABC hydrogels is lower than 19 KJ/mol, which is less than 30% of the literature value calculated using a similar method, implying a significantly fast bond exchange.²¹ Cromwell et al. reported a similar Eₐ value comparing to ours using NMR kinetic study.¹⁷ Interestingly, Eₐ increased with increasing chitosan concentration from AB₁₅ to AB₁₅C₁ and AB15C₂, but AB₁₅C₄ showed lower Eₐ than AB₁₅C₂. Our hypothesis is that excess chitosan starts to interfere and destabilize the PVA/BA network as hydrogen bonding between chitosan and PVA becomes significant. We believe this can also explain the higher elastic modulus of AB₁₅C₂ comparing to AB₁₅C₄ in FIG. 6b.

A similar trend in dissociation enthalpy

$$( Δ {H}_{diss}^{‡} )$$

and entropy

$$( Δ {S}_{diss}^{‡} )$$

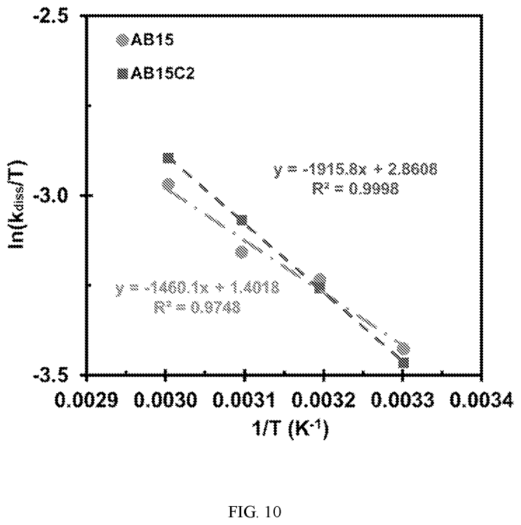

from the Eyring plot (FIG. 10) is observed, as chitosan demands higher transition state energy

$$( Δ {H}_{diss}^{‡} ) ,$$

thus, distavoring boronic ester bond exchange (Table 1). Meanwhile, chitosan disorganizes the transition state

$$( Δ {S}_{diss}^{‡} ) ,$$

benefiting a taster bond breakage and reforming. These two mechanisms compete in Gibbs free energy equation:

$$Δ {G}_{diss}^{‡} = Δ {H}_{diss}^{‡} - T Δ {S}_{diss}^{‡} \qquad ( 2 )$$

As a result, when the temperature is lower than 314 K (41° C.), AB₁₅ has a lower dissociation free energy

$$( Δ {G}_{diss}^{‡} )$$

than AB₁₅C₂ and thus faster bond exchange, and vice versa.

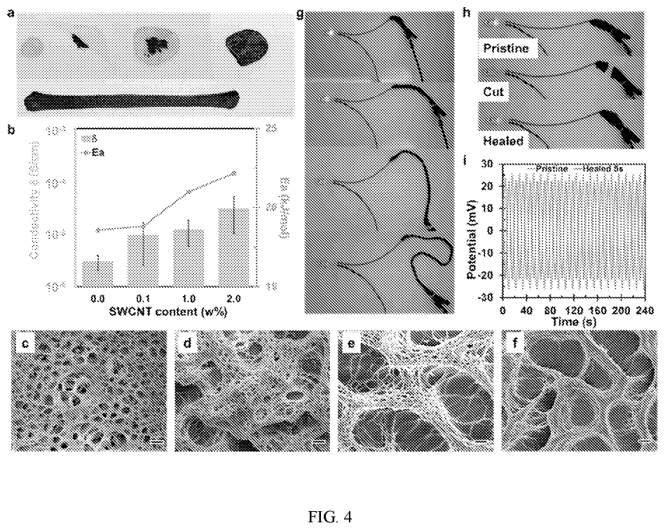

The extreme dynamic nature of ABC hydrogel makes it highly suitable to dissolve or disperse hydrophilic additives simply by kneading. To demonstrate its application as stretchable, self-healed, and conductive hydrogel adhesive, we incorporated single-wall carbon nanotubes (SWCNTs) into AB₁₅C₂ hydrogels. SWCNTs were directly dispersed in AB₁₅C₂ hydrogels followed by hand kneading (FIG. 4a). Within 2 min, 1 wt % (to the total weight of the ABC gel) SWCNT-doped AB₁₅C₂ hydrogel became black and retained its stretchability, self-healing, and adhesive properties. The concentration of SWCNTs was expressed in weight percent comparing to the weight of hydrogels.

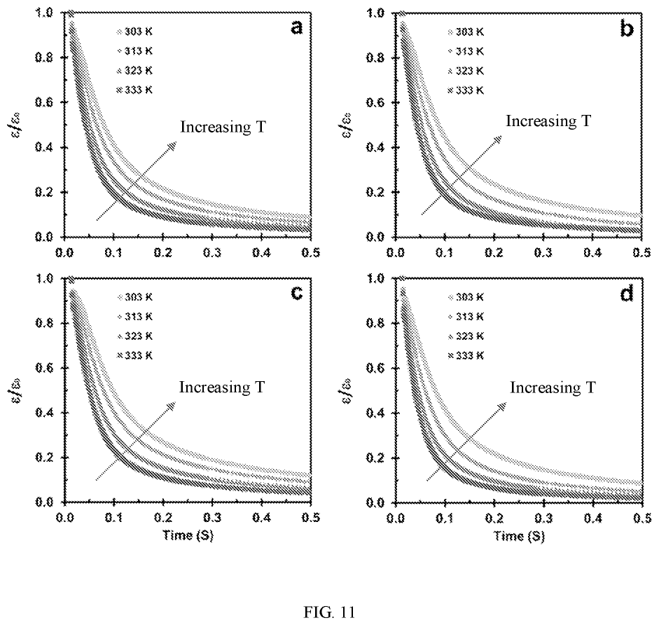

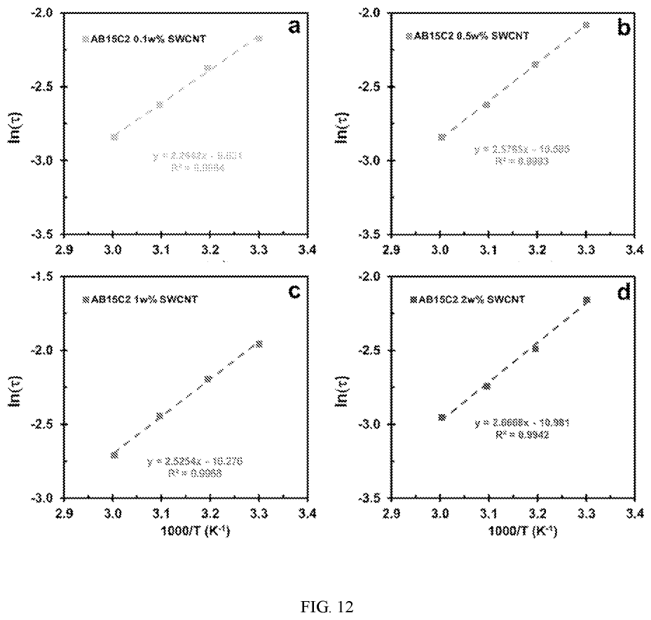

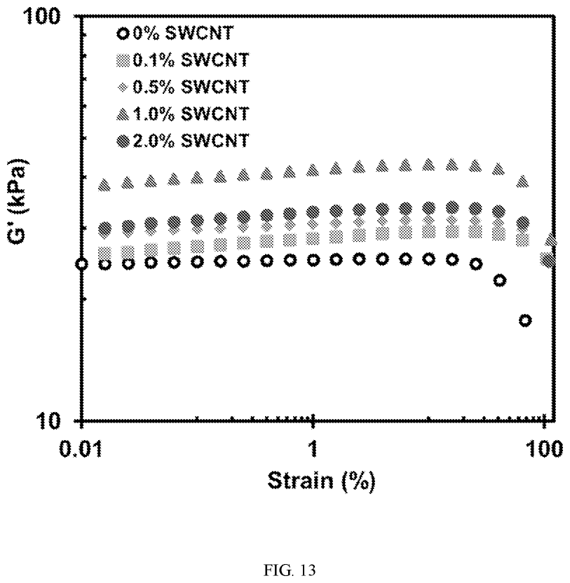

Increasing the loading of SWCNTs resulted in a steady increase in conductivity (FIG. 4b). Pure AB₁₅C₂ hydrogel exhibited a conductivity of 3.0×10⁻⁶ S/cm, which was increased to 9.5×10⁻⁶ S/cm, 1.2×10⁻⁵ S/cm, and 3.1×10⁻⁵ S/cm when adding 0.1 wt %, 1.0 wt % and 2.0 wt % SWCNTs, respectively. The conductivity of SWCNT/ABC hydrogel is on par with previously reported hydrogels with SWCNTs at similar loading.²⁵ Eₐ also increased with higher SWCNT loadings (FIG. 4b, FIG. 11 and FIG. 12), proving the interactions between SWCNTs and ABC hydrogels. The influence of SWCNTs on ABC hydrogel mechanical strength was assessed by rheometer. Introduction of 1 wt % SWCNTs increased the shear storage modulus (G′) by roughly two times compared with that of pristine AB₁₅C₂ hydrogel (FIG. 13). However, as more SWCNTs were introduced, they started to interfere with the ABC network and weaken its mechanical strength. Hence, for the following discussion, we used AB₁₅C₂ with 1 wt % SWCNTs for demonstration. Scanning electron microscopy (SEM) images of the AB₁₅C₂ with increasing loading of SWCNTs demonstrated a shift from a homogeneously porous structure (FIG. 4c) to an increasingly more heterogenous porous network with decreased pore size (FIG. 4c-4f), which is consistent with literature^{26, 27} and further confirmed the interactions between SWCNTs and ABC hydrogels.

As conductive hydrogels, AB₁₅C₂ with 1 wt % SWCNTs featured tunable conductivity via simple stretching and instant conductivity recover from crack or damage. Owing to its inherent adhesion, assembly of the electrical circuit was achieved readily by adhering conductive wires with the hydrogels. The adhesion can withstand extensive stretching. When stretching the hydrogel, the conductivity deceased effectively due to the increased hydrogel length. As a result, the light intensity of the LED light decreased during stretching (FIG. 4g). This process can be readily reversed by compressing the hydrogel back to its original length. When a cut was introduced to the hydrogel, the light went off immediately (FIG. 4h). But the hydrogel self-healed instantly upon contact, leading to an immediate recovery of the light. We applied AC current to quantify the conductivity recovery rate. For the pristine hydrogel, the average maximum potential response Δ|Vmax| was 25.70 mV (FIG. 4i). After only 5 s of self-healing, 87% Δ|Vmax| was recovered to its original conductivity (22.38 mV).

In summary, the ABC hydrogel double network based on PVA/BA dynamically crosslinked matrices and chitosan hydrogen bonded systems have demonstrated great potential as soft materials in cell engineering, wearable electronics, and soft robotics. We demonstrated the extreme stretchability (up to 310) and instant self-healing of the ABC hydrogel owing to its fast stress relaxation and low activation energy of boronic ester bond exchange. The ABC hydrogel also provided adhesion and malleability for easy attachment on complex substrates as well as good reusability. The hydrophilic nature of the ABC hydrogel allowed facile incorporation of various functional additives, such as dyes and conductive particles. Within minutes, SWCNTs was dispersed evenly in the ABC hydrogels and served as conductive components in a working circuit without interfering with the ABC hydrogel's original properties. The conductivity was tunable by simple stretching or compressing while self-healing of the ABC hydrogels exhibited almost instant conductivity recovery (87.1% within 5 s). We envision that the ABC hydrogel will become an ideal material platform for cell engineering, wearable electronics, and soft robotics to provide stretchability, malleability, adherability, reusability, and tear-resistance. The high compatibility of the ABC hydrogel with various hydrophilic additives can further optimize and expand its application.

### Materials and Methods

Materials. Poly(vinyl alcohol) (M_{w} 13,000-23,000 g/mol, 87-89% hydrolyzed), methylene blue and chitosan (low molecular weight) were bought from Sigma-Aldrich and used directly. Boric acid (DNase, RNase and Protease free, 99.5%) was purchased from Acros Organics and used without further treatment. Buffer solution (pH 7, 9, and 11) were prepared from Hydrion buffer capsules with deionized water. SWCNTs (Single Wall/Double Wall Carbon Nanotubes>60 w %, outer diameter 1-4 nm, length 5-30 mm) were acquired from cheaptubes and used without further treatment. LED lights (realUV™ LED strip lights, 365 nm) were purchased from waveform lighting and used directly.

PVA and chitosan of different molecular weight can be used. However, the optimized conditions (such as water content) will change accordingly. We expect higher molecular weight and hydrolysis degree of the PVA will decrease its solubility and thus require higher water content for the gel formation. Higher molecular weight PVA slows down the dynamics of the DCBs while the extra water content (required by the higher molecular weight) mitigates this effect.

ABC hydrogel synthesis. Take the synthesis of AB₁₅C₂ as an example. PVA (7.03 g) was charged into a PTFE container with deionized water (30 g) under vigorous stir at room temperature. After 4 hours, BA (0.47 g) and chitosan (0.6 g) were added directly into the clear and homogeneous PVA solution. After 1 hour, the solution became too viscous to stir for magnetic stir bar. A metal spatula was employed to manually stir the solution for 10 min until a homogeneous gel formed. The gel was kept in closed PTFE container to prevent water evaporation before use. Other AB and ABC gels followed the same procedure with different amount of PVA, BA, and chitosan.

Methylene blue and SWCNTs were incorporated into ABC hydrogels via simple mixing: the weighted solids were directly deposited on the ABC hydrogels. After kneading for 5 min, a homogenous hydrogel was produced.

Rheological measurements. All rheological measurements were conducted on a Discovery Hybrid Rheometer HR 20 with 1000 mm gap. The upper geometry was 40 mm 0.998333° cone plate (UHP Steel).

The strain sweep was conducted from 0.01 to 100% at 1 Hz. The stress relaxation was conducted at 30° C., 40° C., 50°° C., and 60°° C. with 1% initial strain and 300 s relaxation time. Consecutive stress relaxation was performed 5 times in a row at 25° C. with 1% initial strain and 300 s relaxation time. Stress relaxation at different strain was conducted at 25° C. with 0.1%, 0.5%, 1.0% and 5.0% strain.

Self-healing test. Three oscillation amplitude measurements (60 s for each measurement) were conducted consecutively at 1 Hz in discrete sweep mode. The strain amplitudes were 1%, 150%, and 1%, and the temperature was set to 25° C.

Mechanical tests. All tensile tests were conducted on an Instron 5564 Tabletop Materials Testing System in tension mode. The hydrogels were first pressed into rectangular shape (75×25×2 mm). The gels were introduced into a silicone mold (2 mm thick) and sandwiched between two glasses. The glasses were hand-pressed to generate flat gel surfaces, and the gels were carefully cut and released from the mold. As the hydrogels were too soft to be directly mounted on the clamp, glass slides were used as extensions. The hydrogels were used to bridge two glass slides via superglue, and the gap between two glass slides was kept at 5 mm. The resulting assembly was allowed to sit for 5 min before test ensuring thorough adhesion development. The tensile test was performed with pulling rates of 5, 40, 80, and 300 mm/min (FIG. 5a) at room temperature. The glass slides were mounted on the Instron clamps, and the hydrogels were pulled to the maximum extension 505 mm (101, l₀ =5 mm).

The self-healed samples followed the same sample preparation and were then cut into two pieces and reconnected again for 5 s. The healed sample was tested following the same test procedures.

Scanning electron microscopy (SEM) images were taken by JEOL 7500F HRSEM (operated at 5 kV). The hydrogel samples were freeze-dried by immersing in liquid nitrogen, followed by lyophilization. The dried samples were placed on a silicon wafer, evaporated with iridium before SEM imaging.

Circuit assembly. An LED light unit was connected to an external DC power source (12 V) through SWCNT-doped AB₁₅C₂ hydrogels. Electrical wires were directly embedded into the hydrogels without further treatment. The adhesion provided by the hydrogels was enough to ensure a good contact even under high strain (>10).

### FIGURES

FIGS. 1A-1B. Illustration of AB and ABC hydrogels. (a) Cartoon demonstration of AB (left) and ABC (right) hydrogels with chemical details of boronic ester bond exchange and chitosan hydrogen bonding. (b) Comparison of AB₁₅ (left) and AB₁₅C₂ (right) hydrogels at pH7/9/11.

FIGS. 2A-2G. Extreme stretchability and instant self-healing. (a) Demonstration of AB₁₅C₂ hydrogels stretchability (b) at various pulling rates (5/40/300 mm/min) in tensile. Original gap of tensile samples is 5 mm. (c) Self-healing of AB₁₅C₂ between two separate hydrogels (blue gel dyed with methylene blue) via (d) boronic ester bond exchange in (e) divalent, trivalent, and tetravalent fashion. The healed tensile sample exhibited complete recovery within 5 s in (f) tensile tests and oscillation tests. The strain was set to 1%-150%-1%. Filled symbols: storage modulus G′; Empty symbols: loss modulus G″.

FIGS. 1A-3F. Fast stress relaxation and low boronic ester bond exchange activation energy. (a) Stress relaxation of AB₁₅C₁ under different initial strains: 0.1%, 0.5%, 1%, and 5%. AB₁₅C₁ showed systematically (b) higher initial response stress ϵ₀ and (c) longer time to relax 99% (t99%) of the initial stress than AB₁₅. (d) AB₁₅C₁ under consecutive stress relaxation cycles (1% strain) exhibited minimum hysteresis. (e) Stress relaxation of AB₁₅C₁ at 298 K, 303 K, 313 K, 323 K, and 333 K allowed the measurement of characteristic relaxation time τ and (f) the calculation of boronic ester bond activation energy through Arrhenius equation (dotted line: linear fitting).

FIG. 2A-4F. Conductive ABC hydrogels with SWCNTs. (a) Facile preparation of AB15C₂ hydrogels with 1 wt % SWCNTs as conductive materials (knead for 2 min). (b) Increasing SWCNT concentrations improved conductivity and increase boronic ester bond exchange Eₐ. Different SWCNT concentrations, (c) 0 wt %, (d) 0.1 wt %, (e) 1 wt %, (f) 2 wt %, changed homogeneous porous microstructures to heterogenous porous microstructures. Conductive ABC hydrogels allowed (g) fine-tuning of LED light intensity through stretching and (h) instant conductivity recover (5 s) via self-healing after cut. Quantitative study revealed (e) 87.1% conductivity recovery after 5 s healing.

FIGS. 5A-5B. Illustration of tensile tests of (a) AB₁₅C₂ and AB₅C₂ showed (b) no further enhancement from boric acid crosslinker concentration on hydrogel mechanical properties. Pulling rate: 40 mm/min.

FIGS. 6A-6B. Illustration of tensile tests of ABC hydrogels with various chitosan concentration at (a) 40 mm/min and (b) 300 mm/min pulling rate.

FIGS. 7A-7B. Comparison of the (a) tensile properties of AB₁₅C₂ (b) before and (c) after self-healing for 5 s.

FIG. 8. Dehydration tests of AB₁₅C₂ hydrogels in an open (blue circle) or closed (red square) environment. Open environment: ambient condition; closed environment: scintillation vial with closed cap. The solid lines are to guide the eyes.

FIGS. 9A-9B. Stress relaxation of AB₁₅ (a) under various initial strains and (b) consecutive relaxation cycles (1% initial strain).

FIG. 10. Eyring plot of AB15C₂ and AB₁₅ generated from stress relaxation data to calculate dissociation enthalpy and entropy.

FIGS. 11A-11D. Stress relaxation of AB15C₂ hydrogels with (a) 0.1 wt %, (b), 0.5 wt %, (c) 1 wt %, and (d) 2 wt % of SWCNTs at 303 K, 313 K, 323 K, and 333 K.

FIGS. 12A-12D. Arrhenius equation fitting of AB₁₅C₂ hydrogels with (a) 0.1 wt %, (b), 0.5 wt %, (c) 1 wt %, and (d) 2 wt % of SWCNTs to calculate boronic ester bond exchange activation energy. Dotted line: linear fitting.

FIG. 13. Shear storage modulus (G′) as a function of SWCNT loadings in AB₁₅C₂ hydrogels.

### ASPECTS

The following Aspects are illustrative only and do not limit the scope of the present disclosure or the appended claims. Any part or parts of any one or more Aspects can be combined with any part or parts of any one or more other Aspects.

Aspect 1. A reversibly adhesive composition, comprising: a first network that comprises at least two hydroxyl-bearing chains of a first polymer, the at least two hydroxyl-bearing polymer chains being crosslinked by crosslinks that comprise one or more boronic ester bonds, a second network that comprises at least two hydroxyl-bearing chains of a second polymer, and the second polymer optionally hydrogen bonding to the first polymer.

Aspect 2. The composition of Aspect 1, wherein the boronic ester bonds are derived from reaction between a boric acid or a boronic acid and a hydroxyl of the first polymer.

Aspect 3. The composition of any one of Aspects 1-2, further comprising an amount of boric acid, an amount of a boronic acid, or both.

Aspect 4. The composition of Aspect 3, wherein the boronic acid is one or more of phenylboronic acid, a phenylboronic acid derivative, a diboronic acid, a multiboronic acid, an aromatic boronic acid with a substitution, or any combination thereof.

Aspect 5. The composition of Aspect 3, wherein (i) the first polymer and the (ii) amount of boric acid, an amount of a boronic acid, or both are present in a weight ratio of from about 4:1 to about 50:1, optionally about 15:1.

Aspect 6. The composition of Aspect 5, wherein the composition comprises water, and wherein the second polymer is present, relative to the water, at from about 0.1 to about 5 wt %.

Aspect 7. The composition of any one of Aspects 1-6, wherein the first polymer comprises a polyol.

Aspect 8. The composition of Aspect 7, wherein the polyol comprises polyvinyl alcohol (PVA).

Aspect 9. The composition of any one of Aspects 1-8, wherein the first polymer comprises polyvinyl alcohol (PVA) or polyHEMA, poly(hydroxyethyl methacrylate).

Aspect 10. The composition of Aspect 9, wherein the first polymer comprises polyHEMA.

Aspect 11. The composition of any one of Aspects 1-10, wherein the first polymer comprises a monosaccharide, a disaccharide, an oligosaccharide, or a polysaccharide.

Aspect 12. The composition of any one of Aspects 1-11, wherein the second polymer comprises a monosaccharide, a disaccharide, an oligosaccharide, or a polysaccharide.

Aspect 13. The composition of Aspect 12, wherein the polysaccharide comprises chitosan.

Aspect 14. The composition of Aspect 13, wherein the second polymer comprises chitosan and the first polymer comprises PVA.

Aspect 15. The composition of any one of Aspects 1-14, wherein the composition exhibits, under uniaxial force, an elongation to break of up to about 300 times an original length of the composition. The elongation to break can be, for example, up to about 300 times, up to about 250 times, up to about 200 times, up to about 150 times, up to about 100 times, or even up to about 50 times.

Aspect 16. The composition of any one of Aspects 1-15, wherein the composition exhibits, following application of an initial stress under uniaxial force, relaxation of 99% of the initial stress in less than about 25 seconds.

Aspect 17. The composition of Aspect 16, wherein the composition exhibits, following application of an initial stress under uniaxial force, relaxation of 99% of the initial stress in less than about 8 seconds.

Aspect 18. The composition of any one of Aspects 1-17, wherein the composition exhibits, following application of five cycles of an initial stress under uniaxial force, relaxation of 90% of the initial stress in less than about 20 seconds, in each cycle.

Aspect 19. The composition of any one of Aspects 1-18, wherein the composition exhibits an activation energy of less than about 20 KJ/mol for exchange of the boronic ester bonds. The activation energy can be, for example, less than about 20, less than about 18, less than about 16, less than about 14, less than about 12, less than about 10, less than about 8, less than about 6, less than about 4, or even less than about 2 KJ/mol.

Aspect 20. The composition of any one of Aspects 1-19, further comprising an additive, the additive optionally being a hydrophilic additive.

Aspect 21. The composition of any one of Aspects 1-20, wherein the composition is present as a film.

Aspect 22. The composition of Aspect 21, further comprising a water-impervious packaging within which the film is disposed.

Aspect 23. The composition of any one of Aspects 1-22, wherein the composition is derived from a base composition that comprises (i) the first polymer and (ii) one or both of boric acid and boronic acid, the weight ratio of (i) to (ii) in the composition being from about 50:1 to 4:1, optionally about 15:1. The ratio can be, for example, from 50:1 to 4:1, from 45:1 to 5:1, from 40:1 to 7:1, from 35:1 to 10:1, from 30:1 to 15:1, from 25:1 to 20:1, and all intermediate values and subranges.

Aspect 24. The composition of Aspect 23, wherein the base composition comprises water, and wherein the second polymer is present, relative to the water, at from about 0.1 to about 5 wt %, for example, from about 0.1 to about 5 wt %, from about 0.5 to about 4 wt %, from about 1 to about 3 wt %, or even about 2 wt %.

Aspect 25. The composition of any one of Aspects 1-24, wherein when a first and second portion of the composition are contacted to effect adhesion between the two portions (that is, between the first portion and the second portion) and form a combined portion, combined portion exhibits at least one of a modulus and a stress relaxation profile that is substantially identical to the at least one of a modulus and a stress relaxation profile of the first portion or the second portion.

Aspect 26. The composition of any one of Aspects 1-25, wherein the composition exhibits self-healing across an interface between first and second portions of the composition contacted together so as to give rise to a combined portion, the combined portion exhibiting at least one of a modulus and a stress relaxation profile that is substantially identical to the at least one of a modulus and a stress relaxation profile of the first portion or the second portion.

Aspect 27. The composition of Aspect 26, wherein the self-healing takes place within about 5 seconds of the two portions (that is, the first portion and the second portion) being contacted together.

Aspect 28. A method, comprising contacting a composition according to any one of Aspects 1-27 to an adherend so as to adhere the composition to the adherend.

Aspect 29. The method of Aspect 28, wherein the adherend is a tissue.

Aspect 30. The method of Aspect 29, wherein the tissue is skin tissue, oral tissue, vascular tissue, or any combination thereof.

Aspect 31. The method of any one of Aspects 28-30, further comprising releasing the composition from the adherend, the releasing optionally being effected by hydrating the composition.

Aspect 32. The method of Aspect 31, further comprising re-adhering the composition to the adherend.

Aspect 33. A method, comprising hydrating a composition according to any one of Aspects 1-27 that is adhered to an adherend so as to reduce adhesion between the composition and the adherend.

Aspect 34. The method of Aspect 33, further wherein the hydrating releases the composition from the adherend.

Aspect 35. The method of any one of Aspects 33-34, further comprising re-adhering the composition to the adherend.

Aspect 36. A method, comprising contacting a first and second portion of a composition according to any one of Aspects 1-27 so as to effect adhesion between the two portions and form a combined portion, optionally wherein the combined portion exhibits at least one of a modulus and a stress relaxation profile that is substantially identical to the at least one of a modulus and a stress relaxation profile of the first portion or the second portion.

### REFERENCES

1. Röttger, M.; Domenech, T.; Weegen, R. v. d.; Breuillac, A.; Nicolaÿ, R.; Leibler, L., High-Performance Vitrimers from Commodity Thermoplastics through Dioxaborolane Metathesis. Science 2017, 356 (6333), 62-65.

2. Yuk, H.; Wu, J.; Sarrafian, T. L.; Mao, X.; Varela, C. E.; Roche, E. T.; Griffiths, L. G.; Nabzdyk, C. S.; Zhao, X., Rapid and Coagulation-Independent Haemostatic Sealing by a Paste Inspired by Barnacle Glue. Nat. Biomed. Eng. 2021, 5 (10), 1131-1142.

3. Sun, J.-Y.; Zhao, X.; Illeperuma, W. R. K.; Chaudhuri, O.; Oh, K. H.; Mooney, D. J.; Vlassak, J. J.; Suo, Z., Highly Stretchable and Tough Hydrogels. Nature 2012, 489 (7414), 133-136.

4. Kim, J.; Zhang, G.; Shi, M.; Suo, Z., Fracture, Fatigue, and Friction of Polymers in Which Entanglements Greatly Outnumber Cross-Links. Science 2021, 374 (6564), 212-216.

5. Li, Z.; Hou, Z.; Fan, H.; Li, H., Organic-Inorganic Hierarchical Self-Assembly into Robust Luminescent Supramolecular Hydrogel. Adv. Funct. Mater. 2017, 27 (2), 1604379.

6. Chen, Q.; Zhu, L.; Chen, H.; Yan, H.; Huang, L.; Yang, J.; Zheng, J., A Novel Design Strategy for Fully Physically Linked Double Network Hydrogels with Tough, Fatigue Resistant, and Self-Healing Properties. Adv. Funct. Mater. 2015, 25 (10), 1598-1607.

7. South, A. B.; Lyon, L. A., Autonomic Self-Healing of Hydrogel Thin Films. Angew. Chem. Int. Ed. 2010, 49 (4), 767-771.

8. Nakahata, M.; Takashima, Y.; Yamaguchi, H.; Harada, A., Redox-Responsive Self-Healing Materials Formed from Host-Guest Polymers. Nat. Commun. 2011, 2 (1), 511.

9. Chakma, P.; Konkolewicz, D., Dynamic Covalent Bonds in Polymeric Materials. Angew. Chem. Int. Ed. 2019, 58 (29), 9682-9695.

10. Yang, Y.; Pei, Z.; Li, Z.; Wei, Y.; Ji, Y., Making and Remaking Dynamic 3d Structures by Shining Light on Flat Liquid Crystalline Vitrimer Films without a Mold. J. Am. Chem. Soc. 2016, 138 (7), 2118-2121.

11. Lai, Y.; Kuang, X.; Zhu, P.; Huang, M.; Dong, X.; Wang, D., Colorless, Transparent, Robust, and Fast Scratch-Self-Healing Elastomers Via a Phase-Locked Dynamic Bonds Design. Adv. Mater. 2018, 30 (38), 1802556.

12. Kathan, M.; Kovaříček, P.; Jurissek, C.; Senf, A.; Dallmann, A.; Thunemann, A. F.; Hecht, S., Control of Imine Exchange Kinetics with Photoswitches to Modulate Self-Healing in Polysiloxane Networks by Light Illumination. Angew. Chem. Int. Ed. 2016, 55 (44), 13882-13886.

13. Konieczynska, M. D.; Villa-Camacho, J. C.; Ghobril, C.; Perez-Viloria, M.; Tevis, K. M.; Blessing, W. A.; Nazarian, A.; Rodriguez, E. K.; Grinstaff, M. W., On-Demand Dissolution of a Dendritic Hydrogel-Based Dressing for Second-Degree Burn Wounds through Thiol-Thioester Exchange Reaction. Angew. Chem. Int. Ed. 2016, 55 (34), 9984-9987.

14. Brooks, W. L. A.; Sumerlin, B. S., Synthesis and Applications of Boronic Acid-Containing Polymers: From Materials to Medicine. Chem. Rev. 2016, 116 (3), 1375-1397.

15. Zheng, P.; Mccarthy, T. J., A Surprise from 1954: Siloxane Equilibration Is a Simple, Robust, and Obvious Polymer Self-Healing Mechanism. J. Am. Chem. Soc. 2012, 134 (4), 2024-2027.

16. Cash, J. J.; Kubo, T.; Bapat, A. P.; Sumerlin, B. S., Room-Temperature Self-Healing Polymers Based on Dynamic-Covalent Boronic Esters. Macromolecules 2015, 48 (7), 2098-2106.

17. Cromwell, O. R.; Chung, J.; Guan, Z., Malleable and Self-Healing Covalent Polymer Networks through Tunable Dynamic Boronic Ester Bonds. J. Am. Chem. Soc. 2015, 137 (20), 6492-6495.

18. He, L.; Fullenkamp, D. E.; Rivera, J. G.; Messersmith, P. B., Ph Responsive Self-Healing Hydrogels Formed by Boronate-Catechol Complexation. Chem. Commun. 2011, 47 (26), 7497-7499.

19. Lai, J.-C.; Mei, J.-F.; Jia, X.-Y.; Li, C.-H.; You, X.-Z.; Bao, Z., A Stiff and Healable Polymer Based on Dynamic-Covalent Boroxine Bonds. Adv. Mater. 2016, 28 (37), 8277-8282.

20. Ogden, W. A.; Guan, Z., Recyclable, Strong, and Highly Malleable Thermosets Based on Boroxine Networks. J. Am. Chem. Soc. 2018, 140 (20), 6217-6220.

21. Zhao, Z.-H.; Wang, D.-P.; Zuo, J.-L.; Li, C.-H., A Tough and Self-Healing Polymer Enabled by Promoting Bond Exchange in Boronic Esters with Neighboring Hydroxyl Groups. ACS Mater. Lett. 2021, 3 (9), 1328-1338.

22. Chen, W.-P.; Hao, D.-Z.; Hao, W.-J.; Guo, X.-L.; Jiang, L., Hydrogel with Ultrafast Self-Healing Property Both in Air and Underwater. ACS Appl. Mater. Interfaces 2018, 10 (1), 1258-1265.

23. Gong, J. P.; Katsuyama, Y.; Kurokawa, T.; Osada, Y., Double-Network Hydrogels with Extremely High Mechanical Strength. Adv. Mater. 2003, 15 (14), 1155-1158.

24. Dydo, P.; Turek, M., Chapter 11—Boron Removal Using Ion Exchange membranes. In Boron Separation Processes, Kabay, N.; Bryjak, M.; Hilal, N., Eds. Elsevier: Amsterdam, 2015; pp 249-265.

25. Liu, X.; Kim, J. C.; Miller, A. L.; Waletzki, B. E.; Lu, L., Electrically Conductive Nanocomposite Hydrogels Embedded with Functionalized Carbon Nanotubes for Spinal Cord Injury. New J. Chem. 2018, 42 (21), 17671-17681.

26. Du, Z.; Liu, F.; Xiao, C.; Dan, Y.; Jiang, L., Fabrication of Poly(Vinyl Alcohol)/Sodium Alginate Hydrogel Beads and Its Application in Photo-Fenton Degradation of Tetracycline. J. Mater. Sci. 2021, 56 (1), 913-926.

27. Melo, A.; Borges, D.; Debacher, N., Synthesis and Bharacterization of Poly(Vinyl Alcohol)-Boric Acid Beads from Pva with Several Hydrolysis Degrees. e-Polymers 2007, 7.

1. A reversibly adhesive composition, comprising:
a first network that comprises at least two hydroxyl-bearing chains of a first polymer,
the at least two hydroxyl-bearing polymer chains being crosslinked by crosslinks that comprise one or more boronic ester bonds,
a second network that comprises at least two hydroxyl-bearing chains of a second polymer, and
the second polymer optionally hydrogen bonding to the first polymer.

2. The composition of claim 1, wherein the boronic ester bonds are derived from reaction between a boric acid or a boronic acid and a hydroxyl of the first polymer.

3. The composition of claim 1, further comprising an amount of boric acid, an amount of a boronic acid, or both.

4. The composition of claim 3, wherein the boronic acid is one or more of phenylboronic acid, a phenylboronic acid derivative, a diboronic acid, a multiboronic acid, an aromatic boronic acid with a substitution, or any combination thereof.

5. The composition of claim 3, wherein (i) the first polymer and the (ii) amount of boric acid, an amount of a boronic acid, or both are present in a weight ratio of from about 4:1 to about 50:1, optionally about 15:1.

6. The composition of claim 5, wherein the composition comprises water, and wherein the second polymer is present, relative to the water, at from about 0.1 to about 5 wt %.

7. The composition of claim 1, wherein the first polymer comprises a polyol.

8. The composition of claim 7, wherein the polyol comprises polyvinyl alcohol (PVA).

9. The composition of claim 1, wherein the first polymer comprises polyvinyl alcohol (PVA) or polyHEMA, poly(hydroxyethyl methacrylate).

10. The composition of claim 9, wherein the first polymer comprises poly HEMA.

11. The composition of claim 1, wherein the first polymer comprises a monosaccharide, a disaccharide, an oligosaccharide, or a polysaccharide.

12. The composition of claim 1, wherein the second polymer comprises a monosaccharide, a disaccharide, an oligosaccharide, or a polysaccharide.

13. The composition of claim 12, wherein the polysaccharide comprises chitosan and wherein the first polymer optionally comprises PVA.

14. (canceled)

15. The composition of claim 1, wherein the composition exhibits, under uniaxial force, an elongation to break of up to about 300 times an original length of the composition.

16. The composition of claim 1, wherein the composition exhibits, following application of an initial stress under uniaxial force, relaxation of 99% of the initial stress in less than about 25 seconds.

17. (canceled)

18. The composition of claim 1, wherein the composition exhibits, following application of five cycles of an initial stress under uniaxial force, relaxation of 90% of the initial stress in less than about 20 seconds, in each cycle.

19. The composition of claim 1, wherein the composition exhibits an activation energy of less than about 20 KJ/mol for exchange of the boronic ester bonds.

20. (canceled)

21. The composition of claim 1, wherein the composition is present as a film, and further optionally comprising a water-impervious packaging within which the film is disposed.

22. (canceled)

23. (canceled)

24. (canceled)

25. The composition of claim 1, wherein when a first and second portion of the composition are contacted to effect adhesion between the two portions and form a combined portion, combined portion exhibits at least one of a modulus and a stress relaxation profile that is substantially identical to the at least one of a modulus and a stress relaxation profile of the first portion or the second portion.

26. The composition of claim 1, wherein the composition exhibits self-healing across an interface between first and second portions of the composition contacted together so as to give rise to a combined portion, the combined portion exhibiting at least one of a modulus and a stress relaxation profile that is substantially identical to the at least one of a modulus and a stress relaxation profile of the first portion or the second portion.

27. (canceled)

28. A method, comprising contacting a composition according to claim 1 to an adherend so as to adhere the composition to the adherend.

29. The method of claim 28, wherein the adherend is a tissue.

30. (canceled)

31. The method of claim 28, further comprising releasing the composition from the adherend, the releasing optionally being effected by hydrating the composition.

32. (canceled)

33. (canceled)

34. (canceled)

35. (canceled)

36. (canceled)
